(19) United States
(12) Patent Application Publication
(10) Pub. No.: US 2017/0080013 A1
(43) Pub. Date: Mar. 23, 2017

(54) METHODS AND COMPOSITIONS FOR DERMATOLOGICAL USE COMPRSING HYDROCORTISONE ACETATE AND BIOPOLYMERS

(71) Applicant: APEX LABORATORIES PRIVATE LIMITED, Chennai (IN)

(72) Inventors: Sular Subramaniam Vanangamudi, Chennai (IN); Srinivasan Murali, Chennai (IN); Srinivasan Madhavan, Chennai (IN)

(21) Appl. No.: 15/368,552

(22) Filed: Dec. 2, 2016

Related U.S. Application Data
(63) Continuation-in-part of application No. PCT/IB2016/053260, filed on Jun. 3, 2016.

(30) Foreign Application Priority Data
Jun. 10, 2015 (IN) 2896/CHE/2015

(51) Int. Cl. A61K 31/722; A61K 9/00; A61K 9/70; A61K 31/573; A61K 47/02; A61K 47/14; A61K 47/06; A61K 47/12; A61K 47/18; A61K 9/06; A61K 47/10

(52) U.S. Cl. CPC A61K 31/722; A61K 9/06; A61K 9/0014; A61K 9/7015; A61K 31/573; A61K 47/10; A61K 47/14; A61K 47/06; A61K 47/12; A61K 47/183; A61K 47/02

(57) ABSTRACT

Disclosed are compositions comprising hydrocortisone acetate and a biopolymer, chitosan, in a cream base, wherein the cream base comprises a primary and a secondary emulsifier, a waxy material, a co-solvent, a preservative, an acid, a chelating agent, a buffering agent, and water. The compositions disclosed herein are suitable for the treatment of dermatological conditions including but not limited to healing wounds and treatment of dermatitis.

### CROSS-REFERENCE TO RELATED APPLICATION

This application is a continuation-in-part of PCT/M2016/053260, filed Jun. 3, 2016, which application in turn claims priority from Indian Provisional Application Serial 2896/CHE/2015, filed Jun. 10, 2015, the contents of which are incorporated herein by reference.

### FIELD OF THE INVENTION

This present invention is related to dermatological compositions comprising topical corticosteroids and a biopolymer, wherein said biopolymer comprises chitosan.

### BACKGROUND OF THE INVENTION

The outer layer of skin surrounding the body performs an important protective function as a barrier against infection, and serves as a means of regulating the exchange of heat, fluid and gas between the body and external environment. When skin is removed or damaged by being abraded, burned or lacerated, this protective function is diminished. Areas of damaged skin are conventionally treated with dermatological agents and protected by the application of wound dressings to facilitate wound healing.

Wounds to skin and the underlying tissues of animals may be caused by a multitude of external insults such as friction, abrasion, laceration, burning or chemical irritation. Damage to such tissues may also result from internal metabolic or physical dysfunction, including but not limited to bone protrudence, diabetes, circulatory insufficiencies, or inflammatory processes. Normally tissue damage initiates physiological processes of regeneration and repair. In broad terms, this process is referred to as the wound healing process.

Wound healing, or wound repair, is the body's natural process of regenerating dermal and epidermal tissue. The wound healing process is normally uneventful and may occur regardless of any intervention, even in the case of acute or traumatic wounds. However, in certain situations where an underlying metabolic condition or perpetual insult such as pressure is a contributing factor, the natural wound healing process may be retarded or completely arrested, resulting in a chronic wound. When an individual is wounded, a set of complex biochemical events takes place in a closely orchestrated cascade to repair the damage.

The wound healing process progresses through distinct stages leading to the eventual closure, and restoration of the natural function of the tissues. Injury to the skin initiates an immediate vascular response characterized by a transient period of vasoconstriction, followed by a more prolonged period of vasodilation. Blood components infiltrate the wound site, endothelial cells are released, exposing fibrillar collagen, and platelets attach to exposed sites. As platelets become activated, components are released which initiate events of the intrinsic coagulation pathway. At the same time, a complex series of events trigger the inflammatory pathways generating soluble mediators to direct subsequent stages of the healing process.

Wound healing is a complicated process that recruits at least four distinct cell types. Though the process is continuous, it is commonly referred to as occurring in “phases.” The main phases of wound healing include coagulation, which begins immediately after injury; inflammation, which initiates shortly thereafter; a migratory and proliferate process, which begins within days and includes the major processes of healing and a remodeling process, which may last for up to a year and is responsible for scar tissue formation and development of new skin.

Coagulation performs its function of hemostasis, initiating healing and leaving behind messengers that bring on an inflammatory process Inflammation protects the wound from infection and leaves behind its own set of messengers, important signals that bring on the migration and proliferation of macrophages, lymphocytes, fibroblasts, keratinocytes and endothelial cells. In the next phase fibroblasts become dominant and a collagenous matrix is deposited. Finally, there is a remodeling process that aims to restore full and normal structure. Each of these components plays a specific and irreplaceable role in the continuum of healing. A delay in, or absence of any one can result in a prolongation or even a prohibition of healing.

Wound healing is a multifaceted physiological process affected by several factors. These include local factors (growth factors, edema and ischemia, low oxygen tension, and infection), regional factors (arterial insufficiency, venous insufficiency and neuropathy), systemic factors (inadequate perfusion and metabolic disease) and other miscellaneous factors, such as nutritional state, preexisting illnesses, exposure to radiation therapy and smoking. In general, chronic wounds may be managed by preventing or medically treating infections through debridement and occlusive dressings. For wounds that are unresponsive to such interventions, the use of skin replacements may be a viable option.

Given the complex interplay of multiple phases and components in wound healing, it is not surprising that many factors affecting the healing process have been identified. Recognizing and understanding such factors may lead to improved clinical management of recalcitrant or chronic wounds. Patients with risk factors for wound healing may be identified and treated more aggressively or may be better managed for prevention of infection and/or non-healing wounds. Factors affecting wound healing fall into several categories, based on their source; local, regional or systemic.

Trends in modern medical practices have shown that the wound healing of both acute and chronic wounds may be significantly improved by clinical intervention using methods and materials that optimize wound conditions to support the physiological processes of the progressive stages of wound healing. Key factors in providing the optimal conditions are the prevention of scab formation, the prevention of infection and the maintenance of an optimal level of moisture in the wound bed.

In addition to treatment of wounds, several dermatological conditions exist that require therapeutic attention. Such conditions include, for example, acne and related disorders, bacterial skin infections, skin tumors, bullous diseases, cancers of the skin, cornification disorders, fungal skin infections, hypersensitivity and inflammation, parasitic skin infections, pigmentation disorders, psoriasis, atopic dermatitis (eczema), contact dermatitis, dermatitis herpetiformis, generalized exfoliative dermatitis, seborrheic dermatitis, rosacea, shingles, sweating disorders, vitiligo and viral skin disease. Of particular interest is dermatitis, generally considered an inflammation of the skin that is characterized by skin that may be red, swollen, blistered, scabbed, scaly, oozing, or itchy. Whereas some types of dermatitis are caused by allergies, a majority of dermatitis cases do not have any known causes.

Wound (and dermatological) pharmacology is the study of agents and their actions in a wound environment. Wound pharmacology generally comprises three classes of agents: drugs, biologics and special biologics such as those produced by biotechnology. Currently available treatments for both topical and systemic treatment of dermatological issues typically employ corticosteroids in a base component.

There continues to be a need for improved therapeutics that not only address wound healing and repair, but also treat pain associated with dermatological problems, and therapeutics that reduce inflammation, infection, scarring and overall discomfort. There is also a need for therapeutics for dermal conditions that are easily used and applied by patients to accommodate treatment times that may be long and extended. Ideal treatment modalities for wounds and dermal pathologies should be effective and sufficiently straightforward so that a high degree of patient compliance is achieved. Furthermore, there is a need for improved therapeutics requiring a simple and relatively short duration of administration. There is also a need for effective topical treatment of dermatological conditions wherein the compositions enable successful penetration of the active agent, preferably, effective treatments include the penetration, accumulation and maintenance of an effective concentration of active agent at the site of the wound or skin lesion. Compositions are also needed that are effective for treatment pathological conditions in the skin and dermal structures.

### SUMMARY OF THE INVENTION

Disclosed herein are novel methods and compositions comprising hydrocortisone acetate and a biopolymer in a cream base, wherein the cream base comprises a primary and a secondary emulsifier, a waxy material, a co-solvent, a preservative, an acid, a chelating agent, a buffering agent, and water. In certain aspects, the biopolymer comprises a chitosan component. In certain aspects, the chitosan component comprises an unbranched binary polysaccharide consisting of two units N-acetyl-D-glucosamine and D-glucosamine used for the treatment of skin regeneration and rejuvenation and wound healing.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the formation of a film when using the formulation of the present invention.

### DETAILED DESCRIPTION

Before the present compounds, compositions, articles, devices, and/or methods are disclosed and described, it is to be understood that they are not limited to specific synthetic methods or specific pharmacology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

1. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

2. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

3. In this specification and in the claims which follow, reference will be made to a number of terms which shall be defined to have the following meanings:

4. “Optional” or “optionally” means that the subsequently described event or circumstance may or may not occur, and that the description includes instances where said event or circumstance occurs and instances where it does not.

5. Throughout this application, various publications may be referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

6. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular formulation is disclosed and discussed and a number of modifications that can be made to a number of active agents including the biopolymer are discussed, specifically contemplated is each and every combination and permutation of the formulation and the modifications that are possible unless specifically indicated to the contrary. Thus, if a class of active agents A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

7. The present invention comprises the use of biopolymers, including, but not limited to chitin, chitosan, chitosan derivatives chitosan related materials both naturally occurring and synthetically produced.

8. Chitosan is a biopolymer with skin regeneration and rejuvenation properties due to its unique physical nature. Chitosan acts as a biocatalyst in accelerating wound healing. Due to its positive charge it couples with negatively charged blood cells and aids in clotting of blood. Chitosan also contributes to controlling microbial mobility because of its charge and prevents spread of infections. As a micro-film forming biomaterial, chitosan helps in reducing the width of a wound, controls the oxygen permeability at the wound site, and absorbs wound discharge, which is very much essential for faster wound healing. It also reduces itching by providing a soothing effect.

9. Chitosan is an un-branched binary polysaccharide consisting of two units N-Acetyl-D-glucosamine and D-glucosamine linked in β (1, 4) manner. The chemical name of chitosan is Poly-β-(1, 4)-2-Amino-2-deoxy-D-glucose. In certain aspects, chitosan is used as a film forming, mucoadhesive and viscosity-increasing agent. In certain other aspects, chitosan is also used as a binder and disintegrating agent in tablet formulations. Chitosan generally absorbs moisture from the atmosphere or environment and the amount absorbed typically depends upon the initial moisture content, temperature and relative humidity of the environment. Chitosan is regarded as a non-toxic and non-irritant material. It is biocompatible with both healthy and infected skin and has been shown to be biodegradable.

10. In certain aspects, chitosan is produced commercially by deacetylation of chitin, which is the structural element in the exoskeleton of crustaceans (including but not limited to crabs, shrimp, lobsters, hill, woodlice, and barnacles, i.e. members of the Pancrustacia claude) and cell walls of fungi. The degree of deacetylation (% DD) can be determined by NMR spectroscopy, and the % DD in commercial chitosans ranges from 60 to 100%. On average, the molecular weight of commercially produced chitosan is between 3800 and 20,000 Daltons. A common method for the synthesis of chitosan is the deacetylation of chitin using sodium hydroxide in excess as a reagent and water as a solvent. The reaction occurs in two stages under first-order kinetic control. Activation energy for the first step is higher than the second; its value is an estimated 48.76 kJ/mol at 25-120 degrees C. This reaction pathway, when allowed to go to completion (complete deacetylation) yields up to 98% product.

11. The amino group in chitosan has a pKa value of approximately 6.5, which leads to a protonation in acidic to neutral solution with a charge density dependent on pH and the % DA-value. This makes chitosan water-soluble and a bioadhesive which readily binds to negatively charged surfaces such as mucosal membranes. In certain novel embodiments of the present invention, chitosan enhances the transport of pharmaceutical agents across epithelial surfaces, and is biocompatible and biodegradable. Purified quantities of chitosans are suitable for biomedical applications.

12. In certain novel embodiments of the present invention, chitosan and its derivatives, such as trimethylchitosan (where the amino group has been trimethylated), may be used in nonviral gene delivery. Trimethylchitosan, or quaternised chitosan, has been shown to transfect breast cancer cells, with increased degree of trimethylation increasing the cytotoxicity; at approximately 50% trimethylation, the derivative is the most efficient at gene delivery. Oligomeric chitosan derivatives (3-6 kDa) are relatively nontoxic and have good gene delivery properties.

13. Chitosan's properties allow it to rapidly clot blood, and has been granted approval in the United States and Europe for use in bandages and other hemostatic agents. Chitosan hemostatic products also reduce blood loss in comparison to gauze dressings and increase patient survival. Chitosan is hypoallergenic and has natural antibacterial properties.

14. Though not wishing to be bound by the following theory, it is thought that chitosan's hemostatic properties also allow it to reduce pain by blocking nerve endings. Chitosan hemostatic agents are often chitosan salts made from mixing chitosan with an organic acid (such as succinic or lactic acid). The hemostatic agent works by an interaction between the cell membrane of erythrocytes (negative charge) and the protonated chitosan (positive charge) leading to involvement of platelets and rapid thrombus formation. In certain embodiments, chitosan salts can be mixed with other materials to make them more absorbent (such as mixing with alginate), or to vary the rate of solubility and bioabsorbability of the chitosan salt. The chitosan salts are biocompatible and biodegradable making them useful as absorbable haemostats. Protonated chitosan is broken down by lysozyme in the body to glucosamine and the conjugate base of the acid (such as lactate or succinate) are substances naturally found in the body.

15. In certain embodiments, the disclosed compositions and methods of the present invention utilize chitosan's properties to allow it to be used in transdermal drug delivery; it is mucoadhesive in nature, reactive (so it can be produced in many different forms), and importantly, has a positive charge under acidic conditions. This positive charge comes from protonation of its free amino groups. Lack of a positive charge means chitosan is insoluble in neutral and basic environments. However, in acidic environments, protonation of the amino groups leads to an increase in solubility. The implications of this are very important to biomedical applications. This molecule uniquely maintains its structure in a neutral environment, but will solubilize and degrade in an acidic environment.

16. As described herein, chitin and chitosan (CS) are biopolymers having immense structural possibilities for chemical and mechanical modifications to generate novel properties, functions and applications especially in biomedical area. However, despite the availability and functionality of chitosan, the actual utilization of chitin has been restricted by its intractability and insolubility until now. The present inventors have discovered and reduced to practice for the first time, novel compositions and methods of using chitin and chitosan for biomedical use, including but not limited to methods of treating a myriad of dermatological conditions.

17. The novel compositions of the present invention comprise the use of corticosteroids, including but not limited to topical corticosteroids well known to those skilled in the art. Such corticosteroids include for example, hydrocortisone, hydrocortisone acetate, cortisone acetate, diflorasone diacetate, tixocortol pivalate, prednisolone, methylprednisolone, prednisone, triamcinolone acetonide, triamcinolone alcohol, mometasone amcinonide, budesonide, desonid, fluocinonide, fluocinolone acetonide, halcinonide, betamethasone, betamethasone dipropionate, betamethasone sodium phosphate, dexamethasone, dexamethasone sodium phosphate, fluocortolone, mometasone furoate, corticosteroid esters, halogenated corticosteroids (hydrocortisone-17-valerate, halometasone, halobestol propionate, alclometasone dipropionate, betamethasone valerate, betamethasone dipropionate, prednicarbate, clobetasone butyrate, clobetasone-17-butyrate, clobetasol propionate clobetasol-17-propionate, fluocortolone caproate, fluocortolone pivalate, fluprednidene acetate) and labile prodrug esters (hydrocortisone-17-butyrate, hydrocortisone-17-aceponate, hydrocortisone-17-buteprate, ciclesonide and prednicarbate). In certain aspects, the present invention comprises the use of inhalable steroids, including but not limited to flunisolide, fluticasone furoate, fluticasone propionate, triamcinolone acetonide, beclomethasone dipropionate, and budesonide. As is known those skilled in the art, certain corticosteroids may be suitable for topical, inhalation, oral, or systemic use including for example, intravenous and parenteral routes.

18. Though not wishing to be bound by the following theory, it is thought that corticosteroids act by the induction of phospholipase A₂ inhibitory proteins, collectively called lipocortins. It is postulated that these proteins control the biosynthesis of potent mediators of inflammation such as prostaglandins and leukotrienes by inhibiting the release of their common precursor Arachidonic acid. Arachidonic acid is released from membrane phospholipids by phospholipase A₂.

19. Topical corticosteroids are classified by potency, ranging from weak to extremely potent. They include weak potent steroids, moderate potent steroids, potent steroids, very potent steroids and extremely potent steroids. The high potency steroids include betamethasone dipropionate, betamethasone valerate, diflorasone diacetate, clobetasol propionate, halobetasol propionate, desoximetasone, diflorasone diacetate, fluocinonide, mometasone furoate, triamcinolone acetonide, etc. low potency topical steroids include desonide, fluocinolone acetate, and hydrocortisone, etc. Topical corticosteroids are used for the relief of the inflammatory and pruritic manifestations of corticosteroid responsive dermatoses.

20. Hydrocortisone acetate is a member of synthetic steroids and is used as anti-inflammatory and antipruritic agent. It has the chemical name 11β, 17-dihydroxy-3, 20-dioxopregn-4-en-21-yl acetate, the molecular formula is C₂₃H₃₂O₆ and Molecular weight 404.5 g/mol. Hydrocortisone acetate is a white to off-white crystalline powder insoluble in water and slightly soluble in alcohol and in chloroform. Hydrocortisone acetate is a low potent corticosteroid indicated for the relief of the inflammatory and pruritic manifestations of corticosteroid responsive dermatoses.

21. Hydrocortisone acetate is also associated with vasoconstrictive properties. Hydrocortisone acetate depresses formation, release, and activity of endogenous mediators of inflammation, including prostaglandins, kinins, histamine, liposomal enzymes, and complement system, and further modifies the body's immune response.

22. Hydrocortisone acetate has been shown to have a wide range of inhibitory effects on multiple cell types (e.g. mast cells, eosinophils, neutrophils, macrophages and lymphocytes) and mediators (e.g. histamine, eicosanoids, leukotrienes, and cytokines) involved in inflammation and in the asthmatic response. These anti-inflammatory actions of corticosteroids may contribute to their efficacy in asthma and in skin lesions.

23. As would be evident to one skilled in the art, the extent of percutaneous absorption of topical corticosteroids is determined by many factors including, but not limited to, the vehicle, the integrity of the epidermal barrier, and the use of occlusive dressings. Topical corticosteroids may be absorbed from normal intact skin, and in addition, inflammation and/or other disease processes in the skin increase percutaneous absorption. Occlusive dressings substantially increase the percutaneous absorption of topical corticosteroids. Accordingly, an aspect of the present invention comprises the use occlusive dressings in combination with the novel compositions described herein as a valuable therapeutic adjunct for treatment of resistant dermatological conditions. Once absorbed through the skin, topical corticosteroids are handled through pharmacokinetic pathways similar to systemically administered corticosteroids. Corticosteroids are bound to plasma proteins in varying degrees, metabolized primarily in the liver and then excreted by the kidneys. Some topical corticosteroids and metabolites are also excreted into bile.

24. The pH value of human skin is somewhere between 4.5 and 6. Newborn baby's skin pH is closer to neutral (pH 7), but it quickly turns acidic. Nature has designed this probably to protect young children's skin, since acidity kills bacteria. As people age, skin becomes more and more neutral, and fewer bacteria is killed, hence the skin becomes weak and problematic. The pH value goes beyond 6 when a person actually has a skin problem or skin disease. In accordance with the foregoing, there is a preference for dermatological compositions to mirror a pH value closer to that of skin of a young adult.

25. The pH of the novel compositions described herein, comprising chitosan with hydrocortisone acetate cream, is in the range from about 3 to 6. In contrast to available ointments, the presently claimed compositions are not greasy and are cosmetically elegant. In addition, because the active compound is preferably in an ionized form, transdermal penetration is more efficient and more effective.

26. The compositions disclosed herein are highly preferred because the design of the formulation enables active drug penetration of the skin resulting in optimum bio-dermal efficacy. The particle size of the active drug plays an important role here: not only must the particle size be such that therapeutic value is maintained, it must also be such that transdermal delivery is optimized. In a preferred aspect, the active drug is available in colloidal or molecular dispersed state. Also this is to be achieved in the safe pH compatible environment of skin (4.0 to 6.0). The novel compositions disclosed herein satisfy the stated parameters by incorporating optimal vehicles or co-solvents for the dissolution or dispersion of the drug. The disclosed compositions of the present invention are highly efficacious due to the pronounced anti-inflammatory and wound healing activity of the novel combination of the active ingredients, which are available in ultramicron-size, colloidal form, which enhance and enable effective skin penetration for therapeutic efficacy.

27. The novel compositions of the present invention are highly effective in protecting skin, regenerating skin, rejuvenating skin, as well controlling superficial wounds. Furthermore, the compositions of the present invention are particularly desirable as they are affordable, non-allergenic, and safe. In an embodiment, the novel compositions of the present invention comprise a unique combination of a topical corticosteroid (such as hydrocortisone), along with a biopolymer (such as chitosan).

28. In an embodiment, hydrocortisone acetate provides rapid relief of pruritus (severe itching). In addition, hydrocortisone acetate is also recommended for severe eczematic eruptions to provide instant relief to patients from itching and burning. Also monotherapy with hydrocortisone acetate assists in avoiding allergenic response to antifungals and antibacterials.

29. The present invention discloses novel and unique compositions comprising combinations of a steroid, hydrocortisone acetate, with a biopolymer, chitosan. This novel combination is highly therapeutically effective as a result of the unique and desirable physical, chemical and therapeutic properties of chitosan with hydrocortisone acetate. Though not wishing to be bound by the following theory, the superior therapeutic value of the compositions herein are thought to be a result of chitosan which functions as a film forming, biocompatible, non-allergenic biopolymer, protecting the skin by acting as a barrier, and hydrocortisone acetate that acts to attenuate inflammation. Until the innovative discoveries by the present inventors, the unique combination of properties such skin protection, inhibiting the mobility of pathogens from one site to another, and other therapeutic advantages had not been realized. The present invention addresses this long felt need by incorporating the use of biopolymers such as chitosan to optimize skin protection (by way of film forming properties), immobilization of pathogenic microbes (due to its cationic electrostatic property) and wound healing.

30. As previously discussed, chitosan is a non-toxic and non-irritant material; it is biocompatible with both healthy and infected skin and has been shown to be biodegradable. In addition, chitosan shares certain chemical characteristics with GlycosAminoGlycans (GAGs), and GAGs like heparin, heparin sulfate, hyaluronic acid and keratin sulfate all are derivatives of 2-amino-2-deoxy-D-glucose which are present in many parts of human body. GAGs are essential building blocks of macromolecular frame work of connective and other tissues. It is believed that fetal wounds are known to heal without scars as a result of fetal skins being rich in hyaluronic acid. Chitosan/Polyglucosamine is structurally similar to hyaluronan and assists in wound healing with minimal scarring. Heparin enhances mitogen by induction and stabilization of fibroblast growth stimulating factor (FGF). Polyglucosamine may promote tissue growth and wound healing by forming complexes with heparin and acting to prolong the half-life of the growth factors.

31. As a film forming biomaterial, chitosan helps in reducing wound diameters and widths, controls oxygen permeability at the site, absorbs wound discharge and gets degraded by tissue enzymes thereby enabling healing at a faster rate. Chitosan also reduces itching by providing a soothing effect, and acts as a moisturizer.

32. The novel compositions disclosed herein are most stable and efficacious at ambient conditions and do not need special temperature control during transportation or storage, thereby making the present invention further desirable and versatile for a variety of uses including decreased maintenance considerations.

33. The present invention comprises novel compositions that not only diminish the possibility of infection, but also addresses the problem of arresting bleeding. Currently available products and therapies are less effective at controlling superficial bleeding and result in secondary and tertiary complications. The present invention simultaneously addresses bleeding, infection control and wound healing.

34. Disclosed herein are compositions comprising hydrocortisone acetate and a biopolymer in a cream base, wherein the cream base comprises a primary and a secondary emulsifier, a waxy material, a co-solvent, a preservative, an acid, a chelating agent, a buffering agent, and water.

35. In an aspect, the composition may further comprise an anti-oxidant, or a humectant, and in an aspect, the hydrocortisone acetate is added in an amount between 0.001% (w/w) and 5% (w/w), between about 0.01% (w/w) and 2% (w/w), or at 1% (w/w).

36. The biopolymer of compositions disclosed herein may comprise chitosan. In certain aspects, the chitosan is described as being US pharmacopeia conformant with regard to its functional excipient category and selected from any grades such as long chain, medium chain and short chain, and may have a molecular weight in the range of 50 kDa to 5000 kDa. In certain aspects, chitosan is added in an amount between 0.01% (w/w) and 2% (w/w) by weight, in an amount from 0.01% (w/w) to 1.5% (w/w), or 0.5% (w/w).

37. The primary and secondary emulsifiers of the compositions disclosed herein are selected from a group comprising cetostearyl alcohol, cetomacrogol-1000, cetyl alcohol, stearyl alcohol, isopropyl myristate, polysorbate-80, Span-80; and wherein the primary and secondary emulsifiers are present in the amount of 1% (w/w) to 25% (w/w).

38. In an aspect, the waxy materials of the presently disclosed compositions are selected from a group comprising white soft paraffin, liquid paraffin, and hard paraffin; and the waxy material may be added in an amount from 5% (w/w) to 30% (w/w).

39. In an aspect, the compositions disclosed herein comprise a co-solvent wherein the co-solvent is selected from a group comprising propylene glycol, hexylene glycol, polyethylene glycol-400; and wherein the co-solvent is added in an amount from about 5% (w/w) to 50% (w/w).

40. In an aspect, the compositions disclosed herein comprise an acid, wherein the acid is selected from a group comprising HCl, H₂SO₄, HNO₃, and lactic acid; and wherein the acid is added in an amount from about 0.005% (w/w) to 1% (w/w).

41. In an aspect, the compositions disclosed herein comprise a preservative, wherein the preservative is selected from a group comprising methylparaben, propylparaben, chlorocresol, potassium sorbate, benzoic acid, phenoxyethanol, and benzyl alcohol; and wherein the preservative is added in an amount from 0.02% (w/w) to 0.5% (w/w).

42. In an aspect, the buffering agent of the presently disclosed compositions is selected from the group comprising disodium hydrogen orthophosphate, sodium hydrogen orthophosphate; wherein in certain aspects, the buffering agent is added in an amount of 0.05% (w/w) to 1% (w/w).

43. In an aspect, the compositions disclosed herein comprise water, wherein the water is added in the range of 20% (w/w) to 75% (w/w), or 35% (w/w) to 60% (w/w).

44. In an aspect, the compositions disclosed herein comprise anti-oxidants, wherein the anti-oxidant is selected from the group comprising butylated hydroxy anisole, butylated hydroxy toluene; wherein the anti-oxidant is added in an amount of 0.001% (w/w) to 5% (w/w).

45. In an aspect, the compositions disclosed herein further comprise a chelating agent, wherein the chelating agent is selected from the group comprising disodium EDTA; and wherein in certain aspects the chelating agent is added in an amount 0.05% (w/w) to 1% (w/w).

46. In an aspect, the compositions disclosed herein further comprise a humectant, wherein the humectant is selected from a group comprising glycerin, propylene glycol, sorbitol; and wherein the humectant is added in an amount of 5% (w/w) to 20% (w/w).

Pharmaceutical Carriers/Delivery of Pharamceutical Products

47. The disclosed compositions may be administered in vivo in a pharmaceutically acceptable carrier. By “pharmaceutically acceptable” is meant a material that is not biologically or otherwise undesirable, i.e., the material may be administered to a subject, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

48. The disclosed compositions may be administered topically, transdermally, extracorporeally, or the like, including topical intranasal administration or administration by inhalant. The exact amount of the compositions required will vary from subject to subject, depending on the species, age, weight and general condition of the subject, the severity of the disorder being treated, its mode of administration and the like. Thus, it is not possible to specify an exact amount for every composition. However, an appropriate amount can be determined by one of ordinary skill in the art using only routine experimentation given the teachings herein.

49. The compositions can be used therapeutically in combination with a pharmaceutically acceptable carrier.

50. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. It will be apparent to those persons skilled in the art that certain carriers may be more preferable depending upon, for instance, the route of administration and concentration of composition being administered.

51. Pharmaceutical carriers are known to those skilled in the art. These most typically would be standard carriers for administration of therapeutic agents to humans, including solutions such as sterile water, saline, and buffered solutions at physiological pH. The compositions can be administered topically. Other compounds will be administered according to standard procedures used by those skilled in the art.

52. The disclosed compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

53. The disclosed compositions may be administered in a number of ways depending on whether local or systemic treatment is desired, and on the area to be treated. Administration may be topically (including ophthalmically, vaginally, rectally, intranasally), or transdermally.

54. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

55. The novel compositions disclosed herein are preferably formulated as as creams or ointments. As used herein, a “cream” is a topical preparation used for application on the skin. Creams are semi-solid emulsions, which are mixtures of oil and water in which APIs (Active Pharmaceutical Ingredients) are incorporated. They are divided into two types: oil-in-water (O/W) creams which compose of small droplets of oil dispersed in a continuous water phase, and water-in-oil (W/O) creams which compose of small droplets of water dispersed in a continuous oily phase. Oil-in-water creams are user-friendly and hence cosmetically acceptable as they are less greasy and more easily washed with water. An ointment is a viscous semisolid preparation containing APIs, which are used topically on a variety of body surfaces. The vehicle of an ointment is known as ointment base. The choice of a base depends upon the clinical indication of the ointment, and the different types of ointment bases include, but are not limited to: hydrocarbon bases, e.g. hard paraffin, soft paraffin, absorption bases, e.g. wool fat, bees wax.

56. Active compounds in cream formulations are available in ionized state, whereas in case of ointments these are present in non-ionized state. Generally, cream formulations are the first choice of the formulators in design and development of topical dosage forms, as cream formulations are cosmetically elegant, and also as the active compound is available in ionized state, the drug can penetrate the skin layer fast which makes the formulation totally patient friendly.

57. Effective dosages and schedules for administering the disclosed compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

58. Following administration of a disclosed composition, such as corticosteroid in combination with a biopolymer, for treating, inhibiting, or preventing a dermatological condition, the efficacy of the composition can be assessed in various ways well known to the skilled practitioner. For instance, one of ordinary skill in the art will understand that the composition, as disclosed herein is efficacious in treating or inhibiting dermatological condition in a subject by observing that the composition reduces inflammation, induces skin repair or reduces scarring.

59. In an aspect, the compositions described herein may be used to treat wound healing.

60. In an aspect, the compositions described herein may be used to treat dermatological conditions including but not limited to acne and related disorders, bacterial skin infections, skin tumors, bullous diseases, cancers of the skin, cornification disorders, fungal skin infections, hypersensitivity and inflammation, parasitic skin infections, pigmentation disorders, psoriasis, atopic dermatitis (eczema), contact dermatitis, dermatitis herpetiformis, generalized exfoliative dermatitis, seborrheic dermatitis, rosacea, shingles, sweating disorders, vitiligo and viral skin disease.

61. The compositions that improve wound repair and alleviate skin problems disclosed herein may be administered prophylactically to patients or subjects who are at risk for dermatological issues such as psoriasis, inflammation etc.

62. It is understood that the compositions disclosed herein have certain functions, such as having antinflammatory or antiinfective effects. Disclosed herein are certain structural requirements for performing the disclosed functions, and it is understood that there are a variety of structures which can perform the same function which are related to the disclosed structures, and that these structures will ultimately achieve the same result.

### C. METHODS OF MAKING THE COMPOSITIONS

63. The compositions disclosed herein and the compositions necessary to perform the disclosed methods can be made using any method known to those of skill in the art for that particular reagent or compound unless otherwise specifically noted.

64. Disclosed herein are methods for making compositions comprising the mixing of hydrocortisone acetate and a biopolymer in a cream base, wherein the cream base comprises a primary and a secondary emulsifier, a waxy material, a co-solvent, a preservative, an acid, a chelating agent, a buffering agent, and water.

65. The method of making the compositions described herein comprises may further comprise the incorporation of an anti-oxidant, or a humectant. In certain aspects, the methods may comprise the use of hydrocortisone acetate added in an amount between 0.001% (w/w) and 5% (w/w), between about 0.01% (w/w) and 2% (w/w), or at 1% (w/w).

66. In certain aspects, the methods described herein comprise the use of a biopolymer, wherein the biopolymer comprises chitosan. In certain aspects, the chitosan is described as being US pharmacopeia conformant with regard to its functional excipient category and selected from any grades such as long chain, medium chain and short chain, and may have a molecular weight in the range of 50 kDa to 5000 kDa. In certain aspects, the chitosan is added in an amount between 0.01% (w/w) and 2% (w/w) by weight, in an amount from 0.01% (w/w) to 1.5% (w/w), or 0.5% (w/w).

67. In an aspect, the methods described herein comprise the use of primary and secondary emulsifiers selected from a group comprising cetostearyl alcohol, cetomacrogol-1000, cetyl alcohol, stearyl alcohol, isopropyl myristate, polysorbate-80, Span-80; and wherein the primary and secondary emulsifiers are present in the amount of 1% (w/w) to 25% (w/w).

68. In an aspect, the methods disclosed herein comprise a waxy material wherein the waxy material is selected from a group comprising white soft paraffin, liquid paraffin, and hard paraffin; and wherein the waxy material is added in an amount from 5% (w/w) to 30% (w/w).

69. In an aspect, the methods disclosed herein comprise the use of a co-solvent selected from a group comprising propylene glycol, hexylene glycol, polyethylene glycol-400; and wherein the co-solvent is added in an amount from about 5% (w/w) to 50% (w/w).

70. In an aspect, the methods disclosed herein comprise the use of an acid, wherein the acid is selected from a group comprising HCl, H₂SO₄, HNO₃, and lactic acid; and wherein the acid is added in an amount from about 0.005% (w/w) to 1% (w/w).

71. In an aspect, the methods disclosed herein comprise the use of a preservative, wherein the preservative is selected from a group comprising methylparaben, propylparaben, chlorocresol, potassium sorbate, benzoic acid, phenoxyethanol, and benzyl alcohol; and wherein the preservative is added in an amount from 0.02% (w/w) to 0.5% (w/w).

72. In an aspect, the buffering agent used in the methods disclosed herein is selected from the group comprising disodium hydrogen orthophosphate, sodium hydrogen orthophosphate; wherein in certain aspects, the buffering agent is added in an amount of 0.05% (w/w) to 1% (w/w).

73. In an aspect, the methods disclosed herein comprise the use of water, wherein the water is added in the range of 20% (w/w) to 75% (w/w), or 35% (w/w) to 60% (w/w).

74. In an aspect, the methods disclosed herein comprise the use of anti-oxidants, wherein the anti-oxidant is selected from the group comprising butylated hydroxy anisole, butylated hydroxy toluene; wherein the anti-oxidant is added in an amount of 0.001% (w/w) to 5% (w/w).

75. In an aspect, the methods disclosed herein further comprise the use of a chelating agent, wherein the chelating agent is selected from the group comprising disodium EDTA; and wherein in certain aspects the chelating agent is added in an amount 0.05% (w/w) to 1% (w/w).

76. In an aspect, the methods disclosed herein further comprise the use of a humectant, wherein the humectant is selected from a group comprising glycerin, propylene glycol, sorbitol; and wherein the humectant is added in an amount of 5% (w/w) to 20% (w/w).

### D. EXAMPLES

77. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Composition-Hydrocortisone Acetate and Chitosan

TABLE 1
Hydrocortisone Acetate (1.0% w/w) + Chitosan 0.5% w/w Cream

| S. No | INGREDIENTS | Quantity in % |
|---|---|---|
| 1 | Hydrocortisone acetate | 1.082 |
| 2 | Chitosan - M | 0.5 |
| 3 | Lactic acid | 0.25 |
| 4 | White soft paraffin | 8.25 |
| 5 | Cetostearyl alcohol | 8.25 |
| 6 | Cetomacrogol- 1000 | 2.5 |
| 7 | Light liquid paraffin | 5.0 |
| 8 | Isopropyl myristate | 5.0 |
| 9 | Methylparaben | 0.2 |
| 10 | Propylparaben | 0.02 |
| 11 | Disodium EDTA | 0.1 |
| 12 | Propylene glycol | 10 |
| 13 | Monosodium phosphate | 0.1 |
| 14 | Purified water | 58.74 |

78. Table 1 provides one embodiment of the present invention including percentage composition of individual components.

79. The composition described in Table 1 is made according to the process outlined in the steps below:
Step 1: Disperse Monosodium Phosphate, Methyl Paraben and Propyl Paraben in required quantity of Purified Water at 70° C. in Vessel 1.
Step 2: Melt White soft paraffin, Cetostearyl alcohol, Cetomacrogol-1000, Light liquid paraffin and Isopropyl Myristate at 70° C. in Vessel 2 and add to the solution obtained in Step 1. Cool the combined mixture to 50° C. under continuous stirring.
Step 3: Disperse Hydrocortisone Acetate in Propylene Glycol and add it to the above cream base prepared in Step 2.
Step 4: Preparation of Chitosan gel: Dissolve Disodium EDTA followed by Chitosan-M in the remaining Purified Water acidified with Lactic Acid in a separate vessel and add to the above base obtained in step 2 at 40° C. Cool the final cream to 25° C.-30° C. with continuous stirring.

80. The compositions claimed herein and prepared for example, according to the percentages provided in Table 1, provide superior therapeutic efficacy as topically applied anti-inflammatory creams with chitosan. The compositions are particularly useful for the treatment of skin inflammation, dermatitis, and allergic conditions. The novel compositions described herein enable the efficient delivery of active therapeutic agents to penetrate intact skin, to improve skin regeneration and rejuvenation, as well as wound healing.

### 2. Example 2

### API Stability—Hydrocortisone Acetate and Chitosan

### Experimental Data

81. API-Stability experiments were carried out (see Tables 2-10 below) using the compositions of the present invention. Tests were carried out to observe the physical appearance of the product, pH and assay of the API over a period of time. Tests were also carried out to assess the stability of the compositions by subjecting the product to stress studies such as autoclave test and oxidative degradation tests. Animal and human healthy subjects were used for assessment for skin inflammatory studies, acute dermal irritation, blood clotting and clinical efficacy studies over a period of time. For the experiments described below, each gram of hydrocortisone acetate cream contained 1.0% (w/w) of hydrocortisone acetate in the finished pharmaceutical product.

### Product: Hydrocortisone Acetate Cream

### Pack: Aluminum Collapsible Tube

### Composition: For Each g: Hydrocortisone Acetate BP 1.0% w/w

TABLE 2
Description Test, Batch No. HAC- 17
Measured parameter: Physical appearance
Method of measurement: Observation by naked eye
Best value of measured parameter: Homogeneous white to off white viscous cream
(C indicates that the results comply with the initial state)

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | Homogeneous | C | C | C | C | — | — | — | — | — |
| 30° C. 65% RH | white to | — | — | C | C | C | C | C | C | C |
| 25° C. 60% RH | off white | — | — | C | C | C | C | C | C | C |
| Temp. cycling | viscous | C | — | — | — | — | — | — | — | — |
| Freeze thaw | cream | C | — | — | — | — | — | — | — | |

TABLE 3
Description Test, Batch No. HAC- 18
Measured parameter: Physical appearance
Method of measurement: Observation by naked eye
Best value of measured parameter: Homogeneous white to off white viscous cream
(C indicates that the results comply with the initial state)

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | Homogeneous | C | C | C | C | — | — | — | — | — |
| 30° C. 65% RH | white to | — | — | C | C | C | C | C | C | C |
| 25° C. 60% RH | off white | — | — | C | C | C | C | C | C | C |
| Temp. cycling | viscous | C | — | — | — | — | — | — | — | — |
| Freeze thaw | cream | C | — | — | — | — | — | — | — | |

TABLE 4
Description Test, Batch No. HAC- 19
Measured parameter: Physical appearance
Method of measurement: Observation by naked eye
Best value of measured parameter: Homogeneous white to off white viscous cream
(C indicates that the results comply with the initial state)

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | Homogeneous | C | C | C | C | — | — | — | — | — |
| 30° C. 65% RH | white to | — | — | C | C | C | C | C | C | C |
| 25° C. 60% RH | off white | — | — | C | C | C | C | C | C | C |
| Temp. cycling | viscous | C | — | — | — | — | — | — | — | — |
| Freeze thaw | cream | C | — | — | — | — | — | — | — | |

TABLE 5
pH Test, Batch No. HAC- 17
Measured parameter: pH
Limit of measured parameter: 4.0-5.5
Method of measurement: Digital pH meter

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 4.48 | 4.41 | 4.39 | 4.41 | 4.43 | — | — | — | — | — |
| 30° C. 65% RH | | 4.38 | 4.37 | 4.38 | 4.38 | 4.36 | 4.41 | 4.36 | 4.42 | 4.31 |
| 25° C. 60% RH | | 4.34 | 4.29 | 4.37 | 4.42 | 4.34 | 4.33 | 4.31 | 4.40 | 4.28 |
| Temp. cycling | | 4.29 | — | — | — | — | — | — | — | — |
| Freeze thaw | | 4.43 | — | — | — | — | — | — | — | — |

TABLE 6
pH Test, Batch No. HAC- 18
Measured parameter: pH
Limit of measured parameter: 4.0-5.5
Method of measurement: Digital pH meter

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 4.47 | 4.42 | 4.39 | 4.37 | 4.41 | — | — | — | — | — |
| 30° C. 65% RH | | 4.39 | 4.45 | 4.41 | 4.40 | 4.38 | 4.37 | 4.39 | 4.41 | 4.24 |
| 25° C. 60% RH | | 4.40 | 4.43 | 4.48 | 4.36 | 4.41 | 4.31 | 4.40 | 4.34 | 4.31 |
| Temp. cycling | | 4.36 | — | — | — | — | — | — | — | — |
| Freeze thaw | | 4.38 | — | — | — | — | — | — | — | — |

TABLE 7
pH Test, Batch No. HAC- 19
Measured parameter: pH
Limit of measured parameter: 4.0-5.5
Method of measurement: Digital pH meter

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 4.46 | 4.47 | 4.43 | 4.39 | 4.43 | — | — | — | — | — |
| 30° C. 65% RH | | 4.41 | 4.37 | 4.43 | 4.42 | 4.36 | 4.41 | 4.38 | 4.41 | 4.08 |
| 25° C. 60% RH | | 4.39 | 4.44 | 4.40 | 4.46 | 4.39 | 4.41 | 4.39 | 4.33 | 4.11 |
| Temp. cycling | | 4.43 | — | — | — | — | — | — | — | — |
| Freeze thaw | | 4.38 | — | — | — | — | — | — | — | — |

TABLE 8
Assay (%) Test, Batch No. HAC- 17
Measured parameter: Assay (%)
Limit of measured parameter: 90-110%
Method of measurement: HPLC method

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 104.50 | 104.36 | 103.90 | 104.12 | 103.97 | — | — | — | — | — |
| 30° C. 65% RH | | — | — | 103.96 | 104.07 | 103.96 | 103.59 | 104.11 | 103.96 | 103.51 |
| 25° C. 60% RH | | — | — | 103.68 | 104.11 | 104.2 | 104.06 | 103.89 | 104.1 | 103.74 |
| Temp. cycling | | 103.72 | — | — | — | — | — | — | — | — |
| Freeze thaw | | 103.97 | — | — | — | — | — | — | — | — |

TABLE 9
Assay (%) Test, Batch No. HAC- 18
Measured parameter: Assay (%)
Limit of measured parameter: 90-110%
Method of measurement: HPLC method

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 104.42 | 104.41 | 104.24 | 104.02 | 103.92 | — | — | — | — | — |
| 30° C. 65% RH | | — | — | 104.14 | 103.84 | 104.08 | 104.11 | 103.96 | 104.09 | 103.88 |
| 25° C. 60% RH | | — | — | 104.26 | 103.97 | 103.83 | 104.18 | 104.16 | 104.15 | 103.95 |
| Temp. cycling | | 103.96 | — | — | — | — | — | — | — | — |
| Freeze thaw | | 103.89 | — | — | — | — | — | — | — | — |

TABLE 10
Assay (%) Test, Batch No. HAC- 19
Measured parameter: Assay (%)
Limit of measured parameter: 90-110%
Method of measurement: HPLC method

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 104.32 | 104.30 | 104.13 | 103.98 | 104.02 | — | — | — | — | — |
| 30° C. 65% RH | | — | — | 104.08 | 103.88 | 104.11 | 104.27 | 103.86 | 104.09 | 103.54 |
| 25° C. 60% RH | | — | — | 104.16 | 103.67 | 103.83 | 104.06 | 104.13 | 104.07 | 103.87 |
| Temp. cycling | | 103.82 | — | — | — | — | — | — | — | — |
| Freeze thaw | | 103.87 | — | — | — | — | — | — | — | — |

TABLE 11

| Sample Number | Mfg. date | Expiry date |
|---|---|---|
| Present Invention | June 2011 | May 2014 |
| Market Sample | May 2011 | April 2013 |

TABLE 12
Autoclave analysis (%) Test
Measured parameter: Assay (%)
Method of measurement: HPLC Method

| | | Analysis - I (%) | | | Analysis - II (%) | | | Average drop of |
|---|---|---|---|---|---|---|---|---|
| | Name of product | | After | | | After | | Analysis - I (%) and |
| S. No | and details | Initial | autoclave | Drop | Initial | autoclave | Drop | Analysis - II (%) |
| 1 | Present Invention | 104.7 | 101.81 | 2.89 | 104.53 | 101.67 | 2.86 | 2.88 |
| 2 | Market Sample | 103.84 | 97.37 | 6.47 | 103.18 | 96.91 | 6.27 | 6.37 |

TABLE 13
Oxidative degradation analysis (%) Test
Measured parameter: Assay (%)
Method of measurement: HPLC Method

| | | Analysis (%) | | |
|---|---|---|---|---|
| | Name of product | | After | |
| S. No | and details | Initial | Oxidation | Degradation in % |
| 1 | Present Invention | 104.32 | 101.68 | 2.64 |
| 2 | Market Sample | 102.78 | 96.73 | 6.05 |

82. Inference from Table 12: The assay results of autoclave analysis (121° C. applied for 15 Minutes) indicate that the commercially available samples of hydrocortisone acetate cream (S. No. 2) show more percentage drop in API content than for the product of the present invention (S. No. 1).

83. Inference from Table 13: The above assay results of oxidative degradation analysis (30% v/v hydrogen peroxide solution over a period of 12 hours) indicate that the market sample of hydrocortisone acetate cream (S. No. 2) shows significant API degradation (indicated by the percentage drop in API content) than for the product of the present invention (S. No. 1).

84. Summary: From the above data, it is evident that the composition of the present invention is stable at ambient conditions, at elevated temperatures and humid conditions of storage. Also the autoclave studies and oxidative degradation studies further confirm the stability of the product. This is a significant advantage over currently available hydrocortisone acetate creams. The stability of the product is further ascertained by the shelf-life prediction of the formulation using Arrhenius plot of degradation employing Nova-LIMS software.

### 3. Example 3

### Application of Compositions

### Method of Application

85. In an embodiment, the compositions (creams) as disclosed herein are applied after thorough cleansing and drying the affected skin area. The compositions are applied in an amount sufficient to cover the affected skin and surrounding area. The compositions may be applied 1-10 times a day, 2-3 times a day, 1-4 times day, or as necessary depending upon the skin conditions for a full treatment period, even though symptoms may have improved. A full treatment period may be determined by one skilled in the art, such as a health care provider, including but not limited to a physician.

Studies

86. Experimental studies were conducted using the presently described compositions (creams) in the laboratory as well as using suitable animal models and in human volunteers. The aspects tested included—film forming, skin inflammatory activity, blood clotting time, and clinical efficacy. These aspects together demonstrate that the present invention is effective in wound healing.

Film Forming Properties:

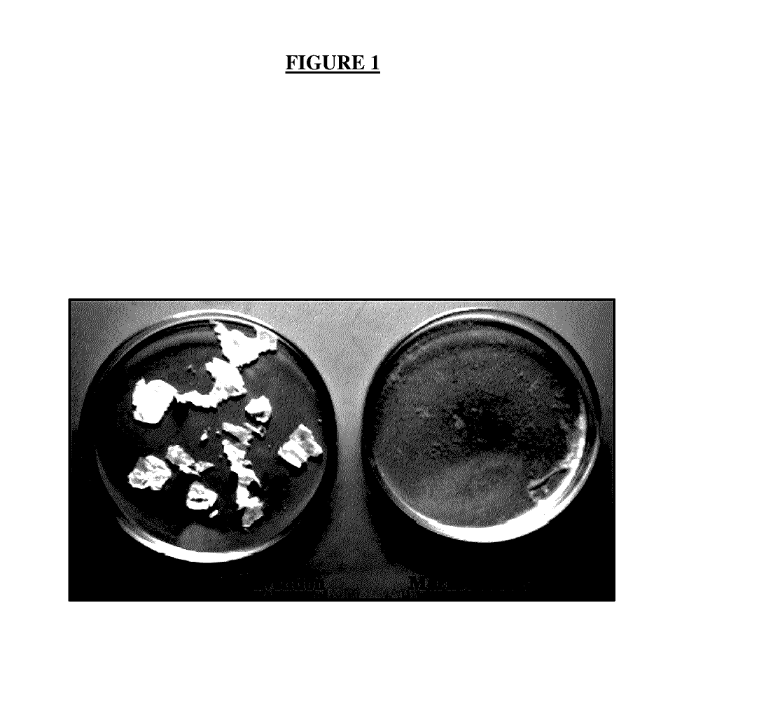

87. It is evident from FIG. 1 that Chitosan does not lose its film forming property in the presence of the excipients used for cream preparations in the present invention. Indeed, chitosan doesn't change its film forming property even in the presently described novel compositions and this ensures that a thin film is formed when cream formulation is applied over the skin. The film formation ensures the moisturizing and soothing effect of the cream and also the even distribution of the active component is ensured when applied over skin. This property particularly valuable when compared to the existing marketed cream formulations.

Skin Inflammatory Study:

Anti-inflammatory activity of the cream of the present invention was determined through animal testing. Croton oil application in to ear of rats has produced 70% edema in control group. It is evident that the hydrocortisone acetate cream of present invention significantly reduced the edema produced by croton oil to 16.32±1.91% compared to market sample which is at 24.51±4.70%.

Study Design

The study was carried out on arachidonic acid mice model (50) and Croton oil ear edema model of rat (50). The study was conducted on five groups of rats (10 in each group) of either sex weighing 150-200 g. The irritant croton oil was prepared by dissolving 4 parts of croton oil, 10 parts of ethanol, 20 parts of pyridine, and 66 parts of ethyl ether. The test compounds were dissolved (5 mg/ml strength) in the croton oil. The control and the test animals received the drug in following manner under ether anesthesia.
Group 1—0.02 ml of croton oil solution, applied on either side of the right ear.
Group 2—0.02 ml of croton oil solution containing dissolved Hydrocortisone Acetate
Cream A—contains 0.25% w/w of Chitosan (apex formulation) 5 mg/ml.
Group 3—0.02 ml of croton oil solution containing dissolved Hydrocortisone Acetate
Cream B—contains 0.50% w/w of Chitosan (apex formulation) 5 mg/ml.
Group 4—0.02 ml of croton oil solution containing dissolved Hydrocortisone Acetate
Cream C—contains 1.25% w/w of Chitosan (apex formulation) 5 mg/ml.
Group 5—0.02 ml of croton oil solution containing dissolved Cutisoft Cream (Ipca Lab) 5 mg/ml.

Four hours after the application the animals were sacrificed under anaesthesia. Both ears were removed and discs of 8 mm diameter are punched. The discs are weighed immediately and the difference in weight between the treated and the untreated ear is indicating the degree of inflammatory edema.

Results

The croton oil application in to ear of rats produced 70% edema in control group. The formulations Hydrocortisone Acetate Cream B, C and Cutisoft significantly reduced the edema produced by croton oil. The highest reduction in edema was by Hydrocortisone Acetate C and Cutisoft. Hydrocortisone Acetate A cream did not produce significant reduction in edema produced by croton oil. (Table A.)

TABLE A
Effect of different formulations of Hydrocortisone
Acetate cream on croton oil induced skin edema

| | Number of | Edema % | | Pro- |
|---|---|---|---|---|
| Group | animals | (mean ± SEM) | P value | tection % |
| Control | 10 | 70.84 ± 3.41 | | |
| Hydrocortisone | 10 | 50.87 ± 9.70 | .195 | 28.19 |
| Acetate Cream A | | | | |
| Hydrocortisone | 10 | 30.42 ± 8.09 | .001 | 57.05 |
| Acetate Cream B | | | | |
| Hydrocortisone | 10 | 16.32 ± 1.91 | .000 | 76.96 |
| Acetate Cream C | | | | |
| CUTISOFT | 10 | 24.51 ± 4.70 | .000 | 65.40 |

P value < 0.05 is considered significant.

Conclusions

In conclusion, it is evident that the formulations Hydrocortisone Acetate Cream B, C and

Cutisoft® significantly reduced the edema induced by croton oil. The formulation Hydrocortisone Acetate C cream is at least as effective as the market available cream namely Cutisoft®. However, all the formulations did not show this effect in arachidonic acid model of skin edema.

Blood Clotting Study:

88. Blood clotting time was observed in both groups of animals, untreated control group and the test group of animals treated with the product of the present invention. Statistically significant decrease in the blood clotting time in treated group animals was observed when compared with that of the control group animals. The mean percent reduction of 62.5% was observed for the blood clotting time using the product of the present invention.

Study Design

89. Nine New Zealand White rabbits (3 Males+6 Female) were acclimatized for seven days in a test room and then randomized into two groups; Group I—Control group (3 Female) and Group-II-Treated group (3 Male+3 Female). Approximately 24 hours prior to initiation of the experiment, an area of about 5 cm on the dorso-lateral hind limb region of both sides were shaved in all experimental rabbits, without making any abrasions with a sterile blade. On the application day, the shaven region was wiped with 70% alcohol. A wound of approximately 3-5 mm length and 1 mm depth was created on both sides of the shaven area in both groups of animals, one by one with sterile disposable lancet. Immediately after the appearance of wound, i.e., when blood started to ooze out, a stopwatch was switched on. Wound in the control group animals was left as such, whereas in the treated group animals, 350 mg of the test substance was applied immediately to the wound region. The stopwatch was stopped once the blood had visibly clotted and the time recorded in the stop clock was noted as clotting time. After the experiment, all the animals were euthanized by CO₂ exposure.

Results

90. The body weight of all animals recorded on the application day showed no statistically significant difference between the control and treated groups by verifying the summary statistics. No clinical signs and mortality was observed individually in animals belonging to control and treated groups. Blood clotting time observed in both control group and treated group animals. The statistical analysis report, for the blood clotting time, showed a highly significant difference between the treated group and the control group animals at 5% level of significant (F=554.27, P<0.0001). Thus a statistically significant decrease in the blood clotting time in hydrocortisone acetate (1% w/w) cream treated group animals when compared with that of the control group animals were observed. No significant difference was observed between the treated and control group animals in other effects like area (between the sides) and gender.

Conclusions

91. From the above experimental results, it may be concluded that the test substance hydrocortisone acetate (1% w/w) Cream was effective in reducing the blood clotting time (62.5%) in New Zealand white rabbits.

Acute Dermal Irritation Study:

92. Skin irritation may be the result of numerous causes, including but not limited to topical exposure to chemicals, drugs, and other toxins or harmful activities such as abrasions or laceration. Depending on the severity of the irritation, and depending on the cause of the irritation, skin damage may be reversible. In designing the appropriate treatment, harmful products may be categorized as irritants or corrosive. The present experimental study was performed to assess the possible hazard likely to arise from exposure of topical formulations to the human skin. Thus a primary skin irritation study was carried out for the composition claimed herein, a newly formulated dermal cream, hydrocortisone acetate cream comprising chitosan to determine its irritant response to the skin after single exposure. From the experimental study it was concluded that the formulation of hydrocortisone acetate cream (invention) score for the primary skin irritation index was 0. Hence, the hydrocortisone acetate cream (invention) was non-irritant and dermal-friendly.

Study Design

93. A total of three female Oryctolaguscuniculus (Rabbit)-New Zealand white (2.110-2.158 Kg) were used. The animals were acclimatized for a minimum of five days and approximately 24 hours before the application of the test item, hairs on the dorsolateral sides of each animal were closely clipped to an area of about 6 cm² on each side without any abrasion. A quantity of 0.5 g of hydrocortisone acetate 1.00% BP w/w cream was applied to the clipped skin area on the left side. The concurrent right untreated side was considered as control area. Both sites were covered with a non-occlusive absorbent gauze patch, which was held in place with non-irritating tape for 4 hours. At the end of 4 hours, the gauze patch was removed and the application site was wiped with lukewarm water without altering the integrity of the epidermis to remove the residual test substance.

94. Initially, the test item was applied to the clipped skin area of one animal and covered with a gauze patch. At the end of 4 hr, as the test item did not cause any dermal reaction, the experiment was repeated in two more animals to confirm the non-irritant response of the test item. Both initial and confirmatory study animals were observed for erythema and edema at 1, 24, 48 and 72 hours following the removal of gauze patch.

Results

95. The body weight of each animal recorded prior to dosing was tabulated in Table A. No skin reactions were recorded at 0 min, 1, 24, 48 and 72 hours after the patch removal in both sides of the initial and confirmatory test animals (Table B).

TABLE B
Individual Body Weight Data

| | | Body weight |
|---|---|---|
| Rabbit No. | Sex | (kg) |
| 1 | Female | 2.135 |
| 2 | Female | 2.110 |
| 3 | Female | 2.158 |

96. None of the animals exhibited any clinical signs of toxicity or mortality. Based on the observations as there were no skin reactions, the Primary Skin Irritation Index of Hydrocortisone Acetate 1.00% BP w/w Cream was calculated as 0.

97. Based on the experimental results, it was concluded that the test item hydrocortisone acetate 1.00% BP w/w Cream is non-irritant to the skin of New Zealand white rabbits.

Clinical Study

98. The study was a randomized, double blind, controlled clinical trial in 20 patients with skin infections (eczema, dermatitis, allergies and rash), inflammatory and pruritic manifestations of corticosteroid responsive dermatosis using hydrocortisone acetate cream of the invention and market sample (Cutisoft (Hydrocortisone acetate 1% w/w) cream of Ipca laboratories limited).

99. The ability of the cream of the present invention to achieve statically significant level of reduction in edema as well as blood clotting time is surprisingly greater than the currently available therapeutics.

Study Design

100. As per the IEC approved protocol, a total of 20 patients were enrolled and completed the trial. Both female and male patients, between the age group of 18-60 years were included for the trial. 20 Patients were randomly assigned to study drug based on randomization schedule. 10 patients received test product and 10 patients received reference product. There were no significant differences between the two groups with respect to their baseline demographic data.

101. Efficacy of the test and reference product were analyzed statistically for the clinical equivalence of the Active Pharmaceutical Ingredient (API). For overall assessment of other skin related parameters such as Visual Analogue Scale (VAS) Score, Global Score Index (GSI), Patient's compliance, Physician's Global Evaluation of Efficacy (PGE), Pruritus Severity Scale etc histograms would be depicted.

102. Based on the qualitative and quantitative assessments in treatment of skin infections (eczema, dermatitis, allergies and rash), inflammatory and pruritic manifestations of corticosteroid responsive dermatosis using hydrocortisone acetate cream of the invention and market sample, the results have been enlisted below: The mean VAS Score for Hydrocortisone Acetate 1% w/w Cream of present invention is 1.8 whereas for market sample is 3 at visit 3.
a. The mean Global Score Index for Hydrocortisone Acetate 1% w/w Cream of present invention is 0.7 whereas for market sample is 1.4 at visit 3.
b. Physician's Global Evaluation Score shows that 80% of the population from Hydrocortisone Acetate 1% w/w Cream of present invention group achieved EXCELLENT results but only 30% achieved EXCELLENT results with market Sample at visit 3.
c. Summary statistics of Patient's Compliance confirmed that 80% of the study population has achieved score zero i.e. absence of signs of itching or indication of pain, from the group that received Hydrocortisone Acetate 1% w/w Cream of present invention but only 30% of study population achieved the same with market Sample at visit 3.
d. The mean Pruritus Severity Scale for Hydrocortisone Acetate 1% w/w Cream of present invention is 0.2 whereas for market sample is 0.6 at visit 3.

Results and Discussions

103. As described herein, the novel compositions of the present invention, comprising chitosan and hydrocortisone acetate, are superior in therapeutic efficacy compared to currently available comparative medicaments. Though not wishing to be bound by the following theory, it is expected that the unique and innovative combination and selection of specific excipients results in achieving the superior results demonstrated herein.

104. The therapeutic impact, as observed from the animal testing and on human volunteers as a result of the novel compositions disclosed herein, wherein said compositions comprise chitosan and hydrocortisone acetate, is shown below in Table 13 by considering various aspects of therapeutic cure of a compromised skin condition:

TABLE 13

| Therapeutic | | Products of the present |
|---|---|---|
| aspect | Market Sample | invention |
| 1. Film forming | None explicitly | Yes (see FIG. 1) |
| property | claimed | |
| 2. Blood | None explicitly | Statistically significant reduction |
| Clotting time | claimed | in clotting time as evidenced by |
| | | pre-clinical animal trials |
| 3. Skin | None explicitly | Expected to immobilize the |
| inflammation | claimed | surface microbes because of the |
| | | cationic charge of Chitosan |
| 4. Acute dermal | Standards as per | The study shows that the formula- |
| irritation study | Existing Products | tion of present invention was non- |
| | | irritant and dermal-friendly while |
| | | applying to the skin |
| 5. Clinical study | — | Clinical equivalent to Market |
| | | product |

105. It is further evident from Table 13, the film forming ability of the chitosan incorporated in the composition enables improved delivery of the API to infected area and results in better functioning, and importantly improved healing.

106. It is evident from the foregoing discussion that the present invention offers the following advantages and unique aspects over the currently available dermaceutical compositions for anti-inflammatory effect and pruritic manifestations of corticosteroid responsive dermatoses of the skin.
The compositions of the present invention include a skin-compatible biopolymer in the form of chitosan which enables enhanced therapeutic outcomes. Such enhanced therapeutic outcomes include, but are not limited to, reduced blood clotting time, and faster relief from skin infection and inflammation.
The compositions of the present invention uniquely incorporate a biopolymer without compromising the stability of the cream matrix and without adversely affecting the functioning of known active pharmaceutical ingredient. The resulting compositions unexpectedly achieve such results through a careful selection of functional excipients to bypass undesirable aspects of physio-chemical compatibility/stability and bio-release.
The compositions of the present invention provide an integrated unit-dose or a single-dose therapy hitherto unavailable in prescription dermaceutical formulations.
The novel compositions of the present invention are adequately stable/efficacious at ambient conditions and do not need special temperature control during transportation/storage.
   The foregoing examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### E. REFERENCES

1. Boucard N, Viton C, Agay D, Mari E, Roger T, Chancerelle Y, Domard A. The use of physical hydrogels of chitosan for skin regeneration following third-degree burns. Biomaterials. 2007; 28(24):3478-88.
2. Okamoto Y 1, Shibazaki K, Minami S, Matsuhashi A, Tanioka S, Shigemasa Y. Evaluation of chitin and chitosan on open wound healing in dogs. J Vet Med Sci. 1995; 57(5):851-4.
3. The United States Pharmacopeia and National Formulary, USP 34-NF 29 Second Supplement Commentary. The United State Pharmacopeial Convention, Rockville. 2011.
4. Gemma Galed, Beatriz Miralles, Inés Paños, Álejandro Santiago, Angeles Heras. N-Deacetylation and depolymerization reactions of chitin/chitosan: Influence of the source of chitin. Carbohydrate Polymers. 2015; 62:316-320.
5. Dai T, Tanaka M, Huang Y Y, Hamblin M R. Chitosan preparations for wounds and burns: antimicrobial and wound-healing effects. Expert Review of Anti-infective Therapy. 2011; 9(7):857-879.
6. The United States Pharmacopeia and National Formulary, USP 34-NF 29. Vol. 2. The United State Pharmacopeial Convention, Rockville. 2011.
7. PDR Staff. Physicians' Desk Reference, Medical Economics Company. 55^{th} Edition Medical Economics Data Production Company, 2000; p.A-350.
8. Lambers H, Piessens S, Bloem A, Pronk H, Finkel P. Natural skin surface pH is on average below 5, which is beneficial for its resident flora. International Journal of Cosmetic Science. 2006; 28(5):359-370.
9. Nguyen N, Hasan S, Caufield L, Ling F S, Narins C R. Randomized controlled trial of topical hemostasis pad use for achieving vascular hemostasis following percutaneous coronary intervention. Catheter. Cardiovasc. Interv. 2007, 69, 801-807.
10. Biagini B, Muzzarelli R A A, Giardino R, Castaldini C. Biological materials for wound healing. In: Brine C J, Sandford P A, Zikakis J P. Advances in chitin and chitosan, Vol. 1. 1992; p 0.6-24.
11. Remington: The Science and Practice of Pharmacy. 19^{th} ed. A. R. Gennaro, Mack Publishing Company, Easton, 1995.

What is claimed is:

1. A composition comprising hydrocortisone acetate and a biopolymer in a cream base, wherein the cream base comprises a primary and a secondary emulsifier, a waxy material, a co-solvent, a preservative, an acid, a chelating agent, a buffering agent, and water.

2. The composition of claim 1, further comprising an anti-oxidant, or a humectant.

3. The composition of claim 1, wherein hydrocortisone acetate is added in an amount between 0.001% (w/w) and 5% (w/w), between about 0.01% (w/w) and 2% (w/w), or at 1% (w/w).

4. The composition of claim 3, wherein the biopolymer comprises chitosan.

5. The composition of claim 4, wherein the chitosan is added in an amount between 0.01% (w/w) and 2% (w/w) by weight, in an amount from 0.01% (w/w) to 1.5% (w/w), or 0.5% (w/w).

6. The compositions of claim 5, wherein the chitosan has a molecular weight in the range of 50 kDa to 5000 kDa.

7. The composition of claim 6, wherein the primary and secondary emulsifiers are selected from a group comprising cetostearyl alcohol, cetomacrogol-1000, cetyl alcohol, stearyl alcohol, isopropyl myristate, polysorbate-80, Span-80; and wherein the primary and secondary emulsifiers are present in the amount of 1% (w/w) to 25% (w/w).

8. The composition of claim 6, wherein the waxy material is selected from a group comprising white soft paraffin, liquid paraffin, and hard paraffin; and wherein the waxy material is added in an amount from 5% (w/w) to 30% (w/w).

9. The composition of claim 6, wherein the co-solvent is selected from a group comprising propylene glycol, hexylene glycol, polyethylene glycol-400; and wherein the co-solvent is added in an amount from about 5% (w/w) to 50% (w/w).

10. The composition of claim 6, wherein the acid is selected from a group comprising HCl, H₂SO₄, HNO₃, and lactic acid; and wherein the acid is added in an amount from about 0.005% (w/w) to 1% (w/w).

11. The composition of claim 6, wherein the preservative is selected from a group comprising methylparaben, propylparaben, chlorocresol, potassium sorbate, benzoic acid, phenoxyethanol, and benzyl alcohol; and wherein the preservative is added in an amount from 0.02% (w/w) to 0.5% (w/w).

12. The composition of claim 6, wherein the buffering agent is selected from the group comprising disodium hydrogen ortho phosphate, sodium hydrogen ortho phosphate; wherein the buffering agent is added in an amount of 0.05% (w/w) to 1% (w/w).

13. The composition of claim 6, wherein the water is purified water, and wherein the water is added in the range of 20% (w/w) to 75% (w/w), or 35% (w/w) to 60% (w/w).

14. The composition of claim 6, further comprising anti-oxidants, wherein the anti-oxidant is selected from the group comprising butylated hydroxy anisole, butylated hydroxy toluene; wherein the anti-oxidant is added in an amount of 0.001% (w/w) to 5% (w/w).

15. The composition of claim 6, further comprising a chelating agent, wherein the chelating agent is selected from the group comprising disodium EDTA; and wherein the chelating agent is added in an amount 0.05% (w/w) to 1% (w/w).

16. The composition of claim 5, further comprising a humectant, wherein the humectant is selected from a group comprising glycerin, propylene glycol, sorbitol; and wherein the humectant is added in an amount of 5% (w/w) to 20% (w/w).

17. A method for making a composition comprising the mixing of hydrocortisone acetate and a biopolymer in a cream base, wherein the cream base comprises a primary and a secondary emulsifier, a waxy material, a co-solvent, a preservative, an acid, a chelating agent, a buffering agent, and water.

18. The method of claim 17, further comprising an anti-oxidant, or a humectant.

19. The method of claim 17, wherein hydrocortisone acetate is added in an amount between 0.001% (w/w) and 5% (w/w), between about 0.01% (w/w) and 2% (w/w), or at 1% (w/w).

20. The method of claim 17, wherein the biopolymer comprises chitosan.

21. The method of claim 20, wherein the chitosan is added in an amount between 0.01% (w/w) and 2% (w/w) by weight, in an amount from 0.01% (w/w) to 1.5% (w/w), or 0.5% (w/w).

22. The method of claim 21, wherein the chitosan has a molecular weight in the range of 50 kDa to 5000 kDa.

23. The method of claim 22, wherein the primary and secondary emulsifiers are selected from a group comprising cetostearyl alcohol, cetomacrogol-1000, cetyl alcohol, stearyl alcohol, isopropyl myristate, polysorbate-80, Span-80; and wherein the primary and secondary emulsifiers are present in the amount of 1% (w/w) to 25% (w/w).

24. The method of claim 22, wherein the waxy material is selected from a group comprising white soft paraffin, liquid paraffin, and hard paraffin; and wherein the waxy material is added in an amount from 5% (w/w) to 30% (w/w).

25. The method of claim 22, wherein the co-solvent is selected from a group comprising propylene glycol, hexylene glycol, polyethylene glycol-400; and wherein the co-solvent is added in an amount from about 5% (w/w) to 50% (w/w).

26. The method of claim 22, wherein the acid is selected from a group comprising HCl, H₂SO₄, HNO₃, and lactic acid; and wherein the acid is added in an amount from about 0.005% (w/w) to 1% (w/w).

27. The method of claim 22, wherein the preservative is selected from a group comprising methylparaben, propylparaben, chlorocresol, potassium sorbate, benzoic acid, phenoxyethanol, and benzyl alcohol; and wherein the preservative is added in an amount from 0.02% (w/w) to 0.5% (w/w).

28. The methods of claim 22, wherein the buffering agent is selected from the group comprising Di Sodium Hydrogen Ortho Phosphate, Sodium Hydrogen Ortho Phosphate; wherein the buffering agent is added in an amount of 0.05% (w/w) to 1% (w/w).

29. The method of claim 22, wherein the water is purified water, and wherein the water is added in the range of 20% (w/w) to 75% (w/w), or 35% (w/w) to 60% (w/w).

30. The method of claim 20, further comprising anti-oxidants, wherein the anti-oxidant is selected from the group comprising butylated hydroxy anisole, butylated hydroxy toluene; wherein the anti-oxidant is added in an amount of 0.001% (w/w) to 5% (w/w).

31. The method of claim 20, further comprising a chelating agent, wherein the chelating agent is selected from the group comprising disodium EDTA; and wherein the chelating agent is added in an amount 0.05% (w/w) to 1% (w/w).

32. The method of claim 20, further comprising a humectant, wherein the humectant is selected from a group comprising glycerin, propylene glycol, sorbitol; and wherein the humectant is added in an amount of 5% (w/w) to 20% (w/w).

33. A method of treating skin problems comprising administering a composition wherein the composition comprises hydrocortisone acetate and chitosan in a cream base, wherein the cream base comprises a primary and a secondary emulsifier, a waxy material, a co-solvent, a preservative, an acid, a chelating agent, a buffering agent, and water.

34. The method of claim 33, wherein the skin problem comprises a wound.

35. The method of claim 33, wherein the skin problem comprises acne, acne-related disorders, bacterial skin infections, skin tumors, bullous diseases, cancers of the skin, cornification disorders, fungal skin infections, hypersensitivity and inflammation, parasitic skin infections, pigmentation disorders, psoriasis, atopic dermatitis, eczema, contact dermatitis, dermatitis herpetiformis, generalized exfoliative dermatitis, seborrheic dermatitis, rosacea, shingles, sweating disorders, vitiligo and viral skin disease.
